(19) United States
(12) Patent Application Publication
(10) Pub. No.: US 2005/0059878 A1
(43) Pub. Date: Mar. 17, 2005

(54) Method of treatment using magnetic resonance and apparatus therefor

(76) Inventor: James Winter, Newtown Square, PA (US)

Correspondence Address: ROBERT S. LIPTON, ESQUIRE, 201 NORTH JACKSON STREET, P. O. BOX 934, MEDIA, PA, 19063-0934, US

(21) Appl. No.: 10/957,476

(22) Filed: Oct. 4, 2004

Related U.S. Application Data
(62) Division of application No. 10/412,913, filed on Apr. 14, 2003.
Application No. 10/412,913 is a continuation of application No. 08/691,949, filed on Aug. 5, 1996, now abandoned.
(60) Provisional application No. 60/002,131, filed on Aug. 10, 1995.

(51) Int. Cl. A61B 5/05

(52) U.S. Cl. 600/410

(57) ABSTRACT

Treatment of malignant tumors or other lesions by localized transfer of radio frequency electromagnetic energy into a portion of the body may be achieved by means of spatially localized magnetic resonance (MR). A magnetic field with appropriate spatial distribution and radio frequency tuned to the resonant frequency unique to the tumor treatment volume will cause selective therapeutic energy deposition or heating within the tumor (hyperthermia). The desired magnetic field distribution for the MR treatment volume may be achieved by means of a main static magnetic field with a superimposed magnetic field to define the treatment volume size and shape, positioned by a gradient magnetic field. Treatment may be enhanced by MR contrast agents and pharmacologic agents. In a preferred embodiment, the invention is incorporated in a magnetic resonance imaging (MRI) scanner modified by the addition of a localizing magnet.

### CROSS-REFERENCE TO RELATED APPLICATIONS

This application is a divisional application and claims the benefit of U.S. patent application Ser. No. 10/412,913 filed Apr. 13, 2003 which is a continuation of U.S. patent application Ser. No. 08/691,949 filed Aug. 5, 1996, which is a Utility Patent Application based upon U.S. Provisional Patent Application No. 60/002,131 filed Aug. 10, 1995 entitled Magnetic Resonance Therapy Scanner.

### NOTICE REGARDING COPYRIGHTED MATERIAL

A portion of the disclosure of the patent document contains materials subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD OF THE INVENTION

This invention relates to Magnetic Resonance specifically to an improved Magnetic Resonance Therapy Scanner™ to selectively deposit energy in a chosen volume of the body or in a lesion for therapeutic purposes.

### DESCRIPTION OF THE PRIOR ART

The prior art includes the use of magnetic resonance for imaging and spectroscopy. These methods use either a uniform magnetic field or a magnetic field with linear variation of field strength sequentially imposed along selected spatial coordinates in conjunction with radio frequency pulse sequences. U.S. Pat. No. 5,423,317 to Iijima and Yamasaki (1995) discloses magnetic resonance imaging, but not therapy. Magnetic resonance is used in the prior art to characterize a chemical or biological sample, or by means of frequency and/or phase encoding to image a volume of a human body by a variety of methods applied point by point, line by line, plane by plane, or volumetrically. This prior art of magnetic resonance does not provide therapeutic energy deposition or hyperthermia. In the prior art of magnetic resonance, elevation of temperature in the human body is considered an undesirable consequence to be minimized for safety reasons and which is due primarily to absorption of radio frequency energy by phenomena other than magnetic resonance.

Prior art radio frequency hyperthermia, diathermy, and ferromagnetic heating rely on methods other than magnetic resonance, even when used with localization by magnetic resonance imaging. The prior art is unable to provide non-invasive precisely localized energy deposition or heating deep within the body. Non-invasive radio frequency hyperthermia heats diffusely over a poorly localized volume. More precise radio frequency hyperthermia requires the invasive placement of a probe or coil within the chosen volume of the body. Ferromagnetic heating requires an invasive procedure with the injection of a foreign substance into the chosen volume. These prior art methods are unable to image, identify, or locate a lesion or volume to be treated and require the use of a separate apparatus for imaging. Such prior art methods may require transfer from the imaging apparatus to the treatment apparatus and may necessitate accurate repositioning that is tedious and difficult to accomplish.

U.S. Pat. No. 5,415,163 to Harms, Flamig, and Griffey (1995) images using magnetic resonance and includes a method of removing a lesion from surrounding tissue using a therapeutic delivery system, for example, laser treatment, but does not use magnetic resonance for delivery of energy to a lesion for therapy. U.S. Pat. No. 5,323,778 to Kandarpa and Jakab (1994) relates to imaging and heating tissues with an invasive probe, through the use of a magnetic resonance imaging radio frequency source to produce heat-generating eddy currents within the tissue, but does not use magnetic resonance for delivery of energy to a lesion for therapy. U.S. Pat. No. 5,378,987 to Ishihara and Sato (1995) uses magnetic resonance for non-invasive temperature measurement, but does not use magnetic resonance for delivery of energy to a lesion for therapy The concept for this invention was derived from Winter's earlier invention of an “Apparatus and Method for Therapeutically Irradiating a Chosen Area Using a Diagnostic Computer Tomography Scanner” (EPO Patent No. 0382560 (1996)). Prior art x-ray and gamma ray imaging and therapy methods require the use of ionizing radiation.

### OBJECTS AND ADVANTAGES

Accordingly, several objects and advantages of a Magnetic Resonance Therapy Scanner are to impart energy for therapeutic purposes; to localize energy deposition; to avoid the use of ionizing radiation; to selectively deposit energy or heat; to treat a chosen volume or lesion; to treat non-invasively; to image, identify, and locate a lesion or chosen volume to be treated; to verify positioning of a body and of a chosen volume of a body during therapy by imaging; and, to image and treat using a single apparatus without any transfer or repositioning.

Additionally, apparatus and methods, including the use of contrast agents, may be used to increase the energy deposition by magnetic resonance, and to decrease the fractional energy deposition by competing mechanisms.

The present invention can be used with a prior art magnetic resonance imaging scanner to add therapeutic capability, and can utilize magnetic resonance imaging and/or spectroscopy to observe and/or control the treatment.

Further objects and advantages of the present invention will become apparent from a consideration of the drawings and ensuing description.

### BRIEF DESCRIPTION OF THE DRAWINGS

The accompanying drawings, which are incorporated in and form a part of the specification, illustrate the embodiments of the present invention and, together with the description, serve to explain the principles of the invention.

FIG. 1 Lesion Being Treated by Magnetic Resonance

FIG. 2 Magnetic Resonance Scanner for Imaging and Therapy

### LIST OF REFERENCE NUMBERLS

10 Magnet Apparatus
12 Spatially Varying Static Magnetic Field
14 Radio Frequency Transmitter
16 Radio Frequency Probe Coil Apparatus
18 Lesion in the Resonance Volume
20 Magnetic Resonance Contrast Agent
22 Magnetic Field Gradient
24 Pharmacologic Agent
26 Computer
28 Image Display
30 Radio Frequency Pulses
32 Positioning Means
34 Localizing Magnetic Field Shaping Apparatus
36 Apparatus to Concentrate Electromagnetic Induction
38 Apparatus to Spatially Localize Electromagnetic Induction
40 Apparatus to Rotate a Spatially Localized Beam of Electromagnetic Induction
42 Main Magnet
44 Magnetic Field Shaping Coils
46 Gradient Coils
48 Probe Coil
50 Radio Frequency Receiver
52 Main Magnetic Field
54 Radio Frequency Apparatus
56 Computer Apparatus
58 Therapeutic Means for Selectively Concentrating Magnetic Resonance
   Summary of the Invention

A Magnetic Resonance Therapy Scanner selectively deposits energy in a chosen volume of a body for therapeutic purposes by varying magnetic field strength spatially while tuning radio frequency pulse sequences to cause magnetic resonance in the chosen treatment volume. Energy deposition is localized by the spatial distribution of the magnetic field and by the choice of radio frequency. Treatment optionally may be further localized by the spatial distribution of the electromagnetic induction, and/or by contrast enhancement, and/or by magnetic field tomography, and/or by electromagnetic induction tomography utilized in a variety of combinations.

Description of the Invention

In describing an embodiment of the invention, specific terminology will be selected for the sake of clarity. However, the invention is not intended to be limited to the specific terms so selected, and it is to be understood that each specific term includes all technical equivalents that operate in a similar manner to accomplish a similar purpose.

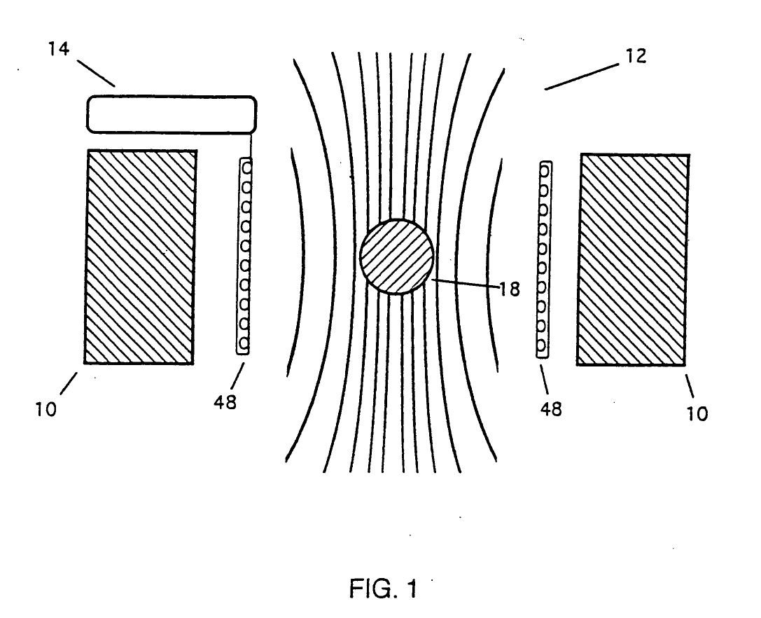

FIG. 1 shows a lesion being treated by magnetic resonance. A magnet apparatus 10 is utilized to produce a spatially varying static magnetic field 12 with position and intensity suitable to allow magnetic resonance to occur only in a chosen volume that corresponds to a lesion to be treated. A radio frequency transmitter 14 produces pulses of suitable frequency oscillating magnetic and electric fields that when applied to probe coil 48 cause magnetic resonance in a lesion in the resonance volume 18 thereby selectively depositing energy in the chosen area or volume of a body for therapeutic purposes.

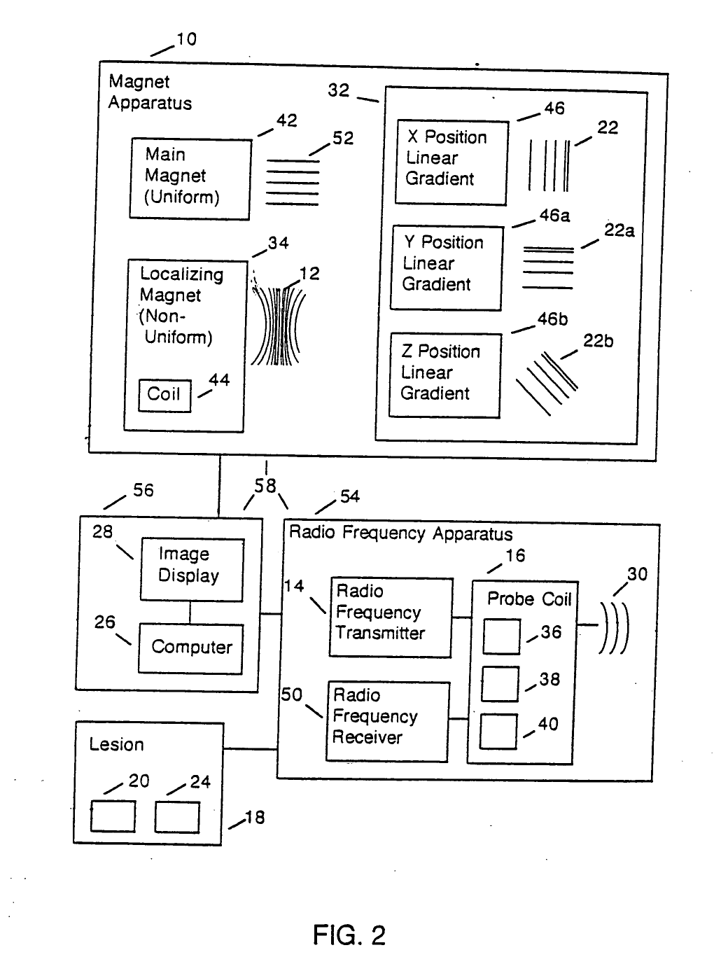

FIG. 2 shows a magnetic resonance scanner for imaging and therapy with apparatus for therapeutically depositing energy, that functions both as a prior art magnetic resonance imaging scanner and also according the present invention as a magnetic resonance therapy scanner that selectively deposits energy in a chosen volume of a body for therapeutic purposes. Magnetic resonance imaging locates the chosen volume of a body containing a lesion by utilizing a magnet apparatus 10 including a main magnet 42 to produce a uniform main magnetic field 52 upon which is superimposed up to three orthogonal linear magnetic field gradients 22 created by utilizing gradient coils 46. Radio frequency pulses 30 created by a radio frequency apparatus 54 using a radio frequency transmitter 14 and radio frequency probe coil apparatus 16 cause magnetic resonance within a body using a variety of pulse sequences while varying the linear magnetic gradients. A radio frequency receiver 50 and radio frequency probe coil apparatus 16 detect magnetic resonance within the body. Computer apparatus 56 including a computer 26 and image display 28 is utilized to control the radio frequency apparatus 54, linear magnetic gradients, and pulse sequences and then to record and process the magnetic resonance data received to produce a sequence of cross sectional or three dimensional images of the body including the lesion to be treated. The computer apparatus 56 including a computer 26 and image display 28 is then utilized to select a chosen volume corresponding to the lesion to be treated.

With further reference to FIG. 2, a magnet apparatus 10 including a uniform main magnetic field 52 created using a main magnet 42, positioning means 32 which may include a movable table top and/or patient couch and linear magnetic field gradients 22 created using gradient coils 46 and a localizing magnetic field shaping apparatus 34 is utilized to produce a correctly positioned spatially varying static magnetic field 12 that may be produced using magnetic field shaping coils 44 and/or other magnet with position and intensity suitable when combined with main magnetic field 52 and positioning means 32 to place the lesion in the resonance volume 18 so as to cause magnetic resonance to occur only in a chosen volume that corresponds to the lesion to be treated. Under control of computer 26, a radio frequency transmitter 14 produces radio frequency pulses 30 of a suitable frequency that when applied to radio frequency probe coil apparatus 16 using probe coil 48 selectively causes magnetic resonance in a lesion in the resonance volume 18 thereby depositing energy in the chosen volume of a body for therapeutic purposes. A magnetic resonance contrast agent 20 may be used to increase the deposition of energy in the chosen volume. Radio frequency probe coil apparatus 16 may deliver electromagnetic induction to the body generally, but may also comprise an apparatus to concentrate electromagnetic induction 36 and/or apparatus to spatially localize electromagnetic induction 38 such as a suitably designed and positioned coil to maximize electromagnetic induction in the chosen volume. Therapeutic means for selectively concentrating magnetic resonance 58 in a chosen volume of a lesion in the resonance volume 18 comprises the combination of magnet apparatus 10, radio frequency apparatus 54, and computer apparatus 56.

With further reference to FIG. 2, radio frequency probe coil apparatus 16 may optionally deliver electromagnetic induction to the body using an apparatus to rotate a spatially localized beam of electromagnetic induction 40 about the chosen volume and/or may rotate an asymmetrical spatially localized magnetic field using the magnetic field shaping coils 44 so as to maximize electromagnetic induction and magnetic resonance selectively in the chosen volume by the methods of intersection and/or tomography. Optionally a pharmacologic agent 24 may be utilized for or to enhance therapeutic purposes. The radio frequency apparatus 54 may optionally utilize radio frequency probe coil apparatus 16 and radio frequency receiver 50 with computer apparatus 56 to detect and/or image physical changes in the chosen volume resulting from the treatment using magnetic resonance imaging and/or spectroscopy, and may control the treatment and/or utilize image display 28 to display the effect of the treatment.

Operation of the Invention

Resonance phenomena occur in suitable materials, such as the human body, in the presence of a particular strength steady magnetic field combined with electromagnetic pulse sequences of the correct frequency determined by the materials and type of magnetic resonance phenomenon but proportional to the strength of the steady magnetic field.

Identification, stereotaxic localization, and outlining of a chosen volume or lesion and verification of positioning may be accomplished by magnetic resonance, computed tomography, or other imaging and by computer graphics and image processing methods, and methods for control of magnetic fields and magnetic gradients to locate a point, line, plane, or volume of magnetic resonance, and for translation and rotation are all well known in the prior art.

The Magnetic Resonance Therapy Scanner imparts energy to a chosen volume by the steps of determining the chosen volume by diagnostic imaging methods such as magnetic resonance imaging well known in the prior art; imparting a non-uniform spatially varying static magnetic field 12 so that the magnetic field strength found at the chosen volume has a suitable value or range which is not found elsewhere in volumes not chosen; and, applying radio frequency pulses 30 at a suitable frequency or range of frequencies of an oscillating magnetic field required to cause magnetic resonance to occur selectively substantially only within the chosen volume thereby resulting in stereotaxic energy deposition or heating of the chosen volume. Such energy deposition or heating may be termed Magnetic Resonance Hyperthermia™.

In the preferred embodiment, the step of determining the chosen volume is performed by a magnetic resonance imaging scanner which has been modified to include the therapeutic capability of the present invention. The step of determining the chosen volume may be performed by use of computer 26 and image display 28, well known in the prior art, whereby a lesion is graphically selected either manually or by automated image processing (for example, by outlining a chosen volume containing a lesion) and the required magnetic resonance operating parameters are calculated to produce the necessary configuration, position, strength, duration, power, current, and frequency of steady and oscillating magnetic fields.

The required spatially varying magnetic field 12 can be created by a magnet apparatus 10 using any of a variety of well known prior art methods. For example a superconducting or resistive electromagnet with suitable shaping coil configuration, design, and current flow or flows through one or multiple suitably configured coils, or a permanent magnet with suitable configuration and pole face geometry can generate a predetermined magnetic field. In such embodiment, the chosen volume would be moved by conventional positioning means 32 such as a movable table to be located at the treatment volume of selected magnetic field strength.

Magnetic Therapy Localization: However, in the preferred embodiment a uniform steady magnetic field is created by the main magnet 42 of a magnetic resonance imager. Additionally, a non-uniform, spatially varying magnetic field 12 of suitable size, shape, and spatial variation is created by localizing magnetic field shaping apparatus 34 using methods well known to practitioners of the prior art in the form of additional energized magnetic field shaping coils 44 consisting of wire wound in the form of an electromagnet provided for this purpose. The coil design and utilization can provide for a particular spatial variation about the center of the magnetic coils or elsewhere, and can provide either a fixed electrical current or a variable direct current used to adjust the volume encompassed. An example is the coil design used in saddle point prior art magnetic resonance imagers. An alternate embodiment uses a permanent magnet or additional permanent magnet with appropriate configuration. This prior art includes shaping of the pole faces by a variety of methods including by casting, machining, by fixed or adjustable rings, or an array of adjustable screw slugs over the poles faces.

Positioning: In a preferred embodiment, the location of the hyperthermia resonance volume may be displaced from the central location within the shaping coils by additionally energizing some or each of the three sets of conventional gradient coils 46 which may also be used for imaging and which each apply a linear magnetic gradient along one of the three orthogonal spatial coordinate directions to displace the treatment volume the required distance, if any, along each coordinate axis to coincide with the location of the chosen volume so as to put the lesion in the resonance volume 18. Such use eliminates any need to mechanically reposition the body, and insures accurate localization of the treatment because geometric errors in the location due to imaging distortions relating to the gradients or other unintended magnetic field variation will be reproduced during therapy thereby canceling any positioning inaccuracy which would otherwise result. Magnetic resonance imaging may also be used to verify continued correct positioning of the patient's body, the chosen volume, and the lesion during treatment.

Probe coil(s) 48 adjacent to or surrounding the body, including those well known in the prior art of magnetic resonance imaging, are used to impart energy to the body by electromagnetic induction using radio frequency pulses 30 derived from a radio frequency transmitter 14.

The radio frequency transmitter 14, of a conventional design well known in the prior art, produces radio frequency oscillations of sufficient power, duration, sequence, and of a precisely delimited frequency or range of frequencies which excite magnetic resonance only within the chosen volume, according to well known physical principles. The power level used may exceed that conventionally used for imaging purposes. The power and duration whether pulsed or continuous may be chosen so as to achieve a calculated or measured temperature rise or energy deposition. The methods of magnetic resonance imaging or spectroscopy may also be used to observe the energy being deposited in the chosen region, as well as to observe resulting physical or biological changes. Magnetic resonance imaging or spectroscopy may thus be used to observe and regulate the magnitude and uniformity of therapy. Thus, heating or energy deposition is continued until a certain degree of physical change in magnetic resonance in the chosen volume is inferred or detected by magnetic resonance imaging or spectroscopy means.

The volume encompassed depends additionally upon the range of radio frequencies applied. By suitably controlling the radio frequency transmitter 14, the range of radio frequencies (the width of the spectral peak) can be used to adjust the volume encompassed. For example, using the magnet apparatus 10 of the Magnetic Resonance Therapy Scanner, a range of magnetic field strengths which exceed a particular threshold value are uniquely located within the chosen volume but not elsewhere. In this case, the range of radio frequencies used is only that range required to excite magnetic resonance in the chosen volume but not elsewhere and corresponding to the exact range of magnetic fields strengths confined to the chosen volume but not found elsewhere.

Scanning of the Magnetic Resonance Therapy Volume (Optional Method): In some embodiments of the invention, in the event that magnetic field shaping coils 44 do not cause the total energy deposition distribution and/or the magnetic resonance treatment volume's size and shape to conform adequately to the full size and exact shape of the lesion or chosen volume or in furtherance of this goal, treatment limited to the chosen volume may be further achieved by continuous or stepwise translation in two or three dimensions during therapy of the non-uniform localizing magnetic field relative to the chosen volume by additionally using positioning means 32 and/or the region of electromagnetic induction using radio frequency probe coil apparatus 16 to achieve a total treatment volume covering the chosen volume which combines smaller volumes treated by sequential scanning of a smaller magnetic resonance volume over the full volume of the chosen volume.

Tomographic Magnetic Resonance Therapy by Rotating an Asymmetrical Magnetic Field about the Chosen Volume (Optional Method): In some other embodiments of the invention, in the event that magnetic field shaping coils 44 do not cause the total energy deposition distribution and/or the magnetic resonance treatment volume's size and shape to conform adequately to the size and shape of the lesion or chosen volume or in furtherance of this goal, treatment limited to the chosen volume may also be further achieved by continuous or stepwise rotation of the shaping magnetic field and/or the region of electromagnetic induction in two or three dimensions during therapy to achieve a tomographic effect. By geometrical superimposition of an asymmetrical rotating volume of magnetic resonance the therapeutic effect will be concentrated only in those locations where the successive magnetic resonance volumes intersect during rotation. Multiple shaping coils or a time varying shaping field can also be used to conform the treatment volume to the chosen volume. An additional method to achieve further spatial selectivity causes a spatially narrow radio frequency beam of electromagnetic induction to intersect the volume of corresponding magnetic field strength to further limit therapy to only a portion of the volume containing a corresponding magnetic field strength. Rotation of such spatially narrow radio frequency beam of electromagnetic induction about the chosen volume may additionally be used to further localize the treatment volume. When also using such a spatially asymmetrical electromagnetic induction, in addition to an asymmetrical magnetic field, such intersection is preferred at an angle (such as at right angles).

Planar Magnetic Resonance Tomographic Therapy by Rotating a Gradient Magnetic Field about the Chosen Volume (Optional Method): Another embodiment of the Magnetic Resonance Therapy Scanner uses a main magnetic field and a linear gradient magnetic field rotating in three dimensions in combination with a radio transmitter tuned to a narrow range of radio frequencies and a probe to impart energy continuously to a chosen volume located within and at the isocenter of such rotation of a finite treatment plane of chosen thickness which by geometric tomography intermittently deposits only a small amount of energy per unit volume outside the chosen volume where the rotating treatment plane thickness does not intersect itself. The thickness of such rotating treatment plane may be varied so as to remain tangent to the borders of and just encompasses the chosen volume in each orientation. The ability of such an embodiment to operate with only linear magnetic gradients as those found on magnetic resonance imaging scanners well known in the prior art also facilitates imaging during therapy. This permits imaging and therapeutically depositing energy to occur simultaneously.

The magnetic field strength and corresponding radio frequency may be chosen so as to maximize magnetic resonance energy deposition or heating, while minimizing competing radio frequency heating effects which are not spatially localized to the chosen volume and which are frequency dependent.

Radio Frequency Localization (Optional Method): Additional measures may be taken to minimize radio frequency heating unrelated to magnetic resonance, and to limit the volume exposed to radio frequency electromagnetic energy so as to further limit heating effects which are not spatially localized to the chosen volume and to maximize magnetic resonance. For example, the probe coil(s) 48 or other probe design or probe placement may be modified so as to concentrate radio frequency pulse energy in the region of the chosen volume. Such methods are well known in the art including but not limited to methods used in radio, microwave, radar design, and the design of probes and antennas (including those for magnetic resonance imaging, radio frequency hyperthermia, and diathermy), resonant cavities and microwave ovens, such as choice of shape and configuration of coils or elements, use of surface coils, parabolic or other directional designs and principles, beam focusing convergent on the chosen volume, use of multiple beams of localized electromagnetic induction directed from a variety of orientations so as to intersect in two or three dimensions at the chosen volume, use of phased array antenna design, quadrature and circular polarization, resonant cavity methods, and the formation of standing waves peaking at the chosen volume. Magnetic resonance scanner pulse sequences may also be adapted so as to maximize magnetic resonance relaxation effects.

The radio frequency is preferentially tuned to the peak resonant frequency. Alternatively, however, the radio frequency may be tuned off the peak of the resonant frequency, just above or just below the frequency of maximum resonance in order to increase energy transfer to the chosen volume, or to limit the energy transfer elsewhere.

In some embodiments of the invention, the probe or other means to deliver radio frequency energy and/or the magnet apparatus or portion thereof may be placed within the body, within an existing body cavity, or blood vessel, or by other means such as by tube, catheter, electrode, needle, or surgical placement.

Contrast Enhancement: Energy deposition or heating may be increased by the use of an administered magnetic resonance contrast agent 20. The effective magnetic moment of electrons of paramagnetic relaxation contrast agents such as gadolinium is approximately 2,000 times larger than the proton magnetic moment. This results in very efficient differential relaxation because it is proportional to the square of the ratio of magnetic moments of the paramagnetic species versus the proton, i.e. 2,000×2,000=4,000,000 times more efficient relaxation. Therefore, paramagnetic magnetic resonance contrast agents such as gadolinium or iron compounds (for example, gadolinium diethylentriamine pentaacetic acid) can be used to markedly enhance the energy deposition or heating effect during magnetic resonance therapy.

Intravascularly injected contrast agents diffuse easily and rapidly, penetrating into the extravascular fluid space in almost all vascularized regions of the human body (except the normal brain), and also slowly penetrate avascular areas, thereby increasing magnetic resonance energy deposition or heating. The exceptional ability of the intact, normal human brain to exclude such contrast agents provides an additional mechanism for selective, localized energy deposition or heating for therapy of brain tumors. Brain tumors often cause blood brain barrier breakdown, thereby concentrating an intravascularly injected magnetic resonance contrast agent 20 such as gadolinium in the chosen volume or lesion in the resonance volume 18.

In addition to administration by intravascular injection, magnetic resonance contrast agents may also be introduced and localized by other well known means such as by direct injection and/or by tube, catheter, or needle. It is additionally possible in the recent prior art for molecular magnetic resonance contrast agents to be made sensitive to specific biological actions and to be locally activated by enzymatic action, exposing a gadolinium or other atom that was enclosed and inactive to magnetic resonance at the time of administration. Other prior art methods can also further increase the naturally occurring blood brain barrier breakdown within tumors.

Pharmaceutical Resonance Therapy: The present invention includes the activation, inactivation, or release of a pharmacologic agent 24, comprising a chemical such as a drug, biological, immunologic agent, or naturally occurring substance within the chosen volume by deposition of energy by means of resonance, including, but not limited to the mechanisms utilized in prior art photo-activation of pharmaceuticals, by heating, by triggering release from a liposome or other encapsulating method, or particle, or by selective excitation at one or more resonance spectral peaks. The trigger of activation or release of an encapsulated or particulate pharmacologic agent at the chosen volume, for example, from a heat sensitive liposome can be assisted by the additional inclusion of a magnetic resonance contrast agent 20 in the wall or interior of each liposome, encapsulant, or particle. Creation of injectable liposomes and particles is well known in the prior art, for example in chemotherapy and nuclear medicine. In a preferred embodiment, magnetic resonance confined to the volume of a tumor causes intravascularly injected liposomes with gadolinium to release a contained chemotherapeutic agent into the tumor.

### CONCLUSION, RAMIFICATIONS, AND SCOPE OF INVENTION

The present invention applies to all of the various magnetic resonance phenomena and apparatus, including conventional and stochastic resonance, oscillatory modes, and chaotic attractors, and including but not limited to nuclear magnetic resonance, electronic magnetic resonance, electron-nuclear double resonance, dipole resonance, quadripole resonance, spin-spin interactions, spin-lattice interactions, and magnetization transfer.

While the term Magnetic Resonance Hyperthermia™ or Magnetic Resonance Therapy by Selective Volume Excitation Hyperthermia literally describes energy deposition consisting of heating produced by resonance, the present invention should be understood to include all forms and manifestation of energy deposition by resonance whether thermal motion, transition to a higher energy state, linear, rotary, vibratory, or otherwise, and regardless of the site whether nuclear, electronic, atomic, molecular, bond, lattice, or otherwise. Such energy deposition or heating has numerous useful applications including in the treatment of medical conditions including tumors, abscesses, musculoskeletal conditions, and other lesions and abnormalities, and in data storage, such as for use with computers, audio, or video recording, and physical, chemical industrial and manufacturing processes and other non-medical applications.

Magnetic Resonance Therapy™ may also be enhanced by adjusting the power level of the radio frequency transmitter 14 because non-linear effects may increase energy transfer.

Spatially localized treatment of malignant or benign masses, tumors, abscesses, vascular malformations, aneurysms, atheromata, and other disorders, obstruction of blood flow, creation of lesions in the nervous system for relief of pain or other neurological disorders, by localized transfer of radio frequency electromagnetic energy into a portion of the body by mechanisms including heating, hyperthermia, radio-coagulation and the production of radio-frequency lesions and other medical and non-medical applications requiring mild, moderate, or extreme localized temperature elevation may be achieved by means of nuclear magnetic resonance radio frequency heating. An appropriately spatially distributed magnetic field and radio transmission tuned to the resonant (Larmour) frequency unique to the treatment volume or a portion thereof will cause selective heating within the treatment volume.

Spatial distribution of the magnetic field may be achieved by point, line, plane or volume methods, including methods used for nuclear magnetic resonance (NMR) imaging, by superconducting magnet, resistive magnet, permanent magnet, or combination, by saddle point methods, by external or internal materials such as ferromagnetic or paramagnetic materials or magnets including by placement using pharmacological localization, localization of particles by the reticuloendothelial system, by monoclonal antibodies, or by other localization methods, by external coil or coils, by internal coil or coils, by coils or substances introduced into the body by tube, catheter, surgical placement, injection, or other means, by electrical current outside, inside or through the body tissues including by external or internal electrode, or by other methods. The desired magnetic field distribution may be achieved in a preferred embodiment by means of a main (B₀) constant magnetic field with superimposed volume defining magnetic field and with additional superimposed gradient magnetic field for positioning the treatment volume. The heating may be achieved by simultaneous heating of an entire selected volume, successively point by point, by successive superimposition of small volumes, including by successively excited points, lines or planes using well known selective excitation methods as practiced in prior art magnetic resonance imaging systems. Alternatively, multiple selectively excited lines all passing two or three dimensionally through the same treatment point can be used to heat the region of said treatment point analogously as currently practiced in stereotactic gamma-ray radiosurgery using the gamma knife.

In a preferred embodiment, the invention is incorporated in a magnetic resonance imaging scanner wherein the imager is used to visually or by automated or semi-automated computer image processing to define and localize the treatment volume or volumes and the main magnetic field and gradient fields are created by the same or by separate magnet apparatus used for imaging and the radio signal used for heating is created by the same or by a separate transmitter and probe coil as that used for imaging.

The invention includes application of the described apparatus and method when used for non-medical applications including but not limited to manufacture, fabrication, curing, bonding, information recording, and other applications.

The invention described above is, of course, susceptible to many variations, modifications and changes, all of which are within the skill of the art. It should be understood that all such variations, modifications, and changes are within the spirit and scope of the invention and of the appended claims. Similarly, it will be understood that it is intended to cover all changes, modifications and variations of the example of the invention herein disclosed for the purpose of illustration which do not constitute departures from the spirit and scope of the present invention. The present invention is intended to be protected broadly within the spirit and scope of the appended claims.

1. An apparatus for therapeutically treating a lesion by generating magnetic resonance in a chosen volume of the lesion comprising:
a) a main magnet having an associated magnetic field;
b) a control computer;
c) at least one magnetic field shaping coil of a type, that in conjunction with the field produced by the main magnet produces a magnetic field of desired strength substantially corresponding and limited to the chosen volume of the lesion, disposed within the magnetic field of the main magnet;
d) at least one radio frequency coil disposed within the magnetic field of the main magnet;
e) a radio frequency transmitter;
f) means for electrically connecting the radio frequency transmitter to the at least one radio frequency coil; and
g) means for electrically connecting the control computer to the at least one magnetic field shaping coil and the radio frequency transmitter
wherein the appropriate radio frequency and magnetic field strength cause magnetic resonance to occur selectively substantially within the chosen volume of the lesion.

2. The apparatus of claim 1 further comprising:
a) at least one magnetic gradient coil disposed within the magnetic field of the main magnet about the at least one magnetic field shaping coil; and
b) means for electrically connecting the control computer to the magnetic gradient coils.

3. The apparatus of claim 2 in which the at least one magnetic gradient coil comprises a means for translating or rotating the magnetic field within the chosen volume.

4. The apparatus of claim 1 further comprising:
a) a radio frequency receiver;
b) means for electrically connecting the radio receiver to the at least one radio frequency coil; and
c) means for electrically connecting the control computer to the radio frequency receiver.

5. The apparatus of claim 4 further comprising:
a) at least one magnetic gradient coil disposed within the magnetic field of the main magnet about the at least one magnetic field shaping coil; and
b) means for electrically connecting the control computer to the at least one magnetic gradient coil.

6. The apparatus of claim 5 in which the plurality of magnetic gradient coils comprise means for translating or rotating the magnetic field within the chosen volume.

7. An apparatus for therapeutically treating a lesion by generating magnetic resonance in a chosen volume of the lesion comprising:
a) means for generating and shaping a magnetic field to produce a magnetic field of desired strength substantially corresponding and limited to the chosen volume of the lesion;
b) a means for generating electromagnetic radiation;
c) a means for delivering electromagnetic radiation; and
d) control means for controlling the means for generating and shaping a magnetic field and for controlling the electromagnetic radiation generating and delivering means
wherein the appropriate electromagnetic radiation and magnetic field strength cause magnetic resonance to occur selectively substantially within the chosen volume of the lesion.

8. The apparatus of claim 7 further comprising:
a) a means for creating magnetic field gradients within the chosen volume of the lesion; and
b) a control means for controlling the means for creating magnetic field gradients within the chosen volume of the lesion.

9. The apparatus of claim 8 in which the means for creating magnetic field gradients within the chosen volume of the lesion comprise a means for translating or rotating the magnetic field within the chosen volume of the lesion.

10. The apparatus of claim 7 further comprising:
a) means for receiving electromagnetic radiation: and
b) a control means for controlling the means for receiving electromagnetic radiation.

11. The apparatus of claim 10 further comprising:
a) a means for creating magnetic field gradients within the chosen volume of the lesion; and
b) a control means for controlling the means for creating magnetic field gradients within the chosen volume of the lesion.

12. The apparatus of claim 11 in which the means for creating magnetic field gradients within the chosen volume of the lesion comprise a means for translating or rotating the magnetic field within the chosen volume of the lesion.

13. An apparatus for therapeutically treating a lesion by generating magnetic resonance in a chosen volume of the lesion comprising:
a) a means for generating a main magnetic field with the chosen volume of the lesion;
b) a means for shaping the magnetic field within the chosen volume of the lesion to produce a magnetic field of desired strength substantially corresponding and limited to the chosen volume of the lesion;
c) means for generating electromagnetic radiation;
d) means for delivering electromagnetic radiation; and
e) control means for controlling the means for generating and shaping the magnetic field and for controlling the electromagnetic radiation generating and delivering means wherein the appropriate electromagnetic radiation and magnetic field strength cause magnetic resonance to occur selectively substantially within the chosen volume of the lesion.

14. The apparatus of claim 13 further comprising:
a) a means for creating magnetic field gradients within the chosen volume of the lesion; and
b) a control means for controlling the means for creating magnetic field gradients within the chosen volume of the lesion.

15. The apparatus of claim 14 in which the means for creating magnetic field gradients within the chosen volume of the lesion comprise a means for translating or rotating the magnetic field within the chosen volume of the lesion.

16. The apparatus of claim 13 further comprising:
a) means for receiving electromagnetic radiation: and
b) a control means for controlling the means for receiving electromagnetic radiation.

17. The apparatus of claim 16 further comprising:
a) a means for creating magnetic field gradients within the chosen volume of the lesion; and
b) a control means for controlling the means for creating magnetic field gradients within the chosen volume of the lesion.

18. The apparatus of claim 17 in which the means for creating magnetic field gradients within the chosen volume of the lesion comprise a means for translating or rotating the magnetic field within the chosen volume of the lesion.
